(19) United States
(12) Patent Application Publication
(10) Pub. No.: US 2010/0106221 A1
(43) Pub. Date: Apr. 29, 2010

(54) WIRELESS COMMUNICATION DEVICE WITH ELECTRIC ACUPUNCTURE FUNCTION

(75) Inventor: Po-Lin Su, Tu-Cheng (TW)

Correspondence Address: PCE INDUSTRY, INC.;ATT. Steven Reiss, 288 SOUTH MAYO AVENUE, CITY OF INDUSTRY, CA, 91789, US

(73) Assignee: HON HAI PRECISION INDUSTRY CO., LTD., Tu-Cheng (TW)

(21) Appl. No.: 12/504,659

(22) Filed: Jul. 16, 2009

(30) Foreign Application Priority Data
Oct. 28, 2008 (CN) 200810305252.X

(51) Int. Cl. A61N 1/00; A61B 17/34

(52) U.S. Cl. 607/60; 606/189

(57) ABSTRACT

A wireless communication device includes a power source, a control unit, a main unit, a conversion unit, a switch unit and an output unit. The control unit is configured for generating a control signal according to predetermined properties for an electric acupuncture and outputting an on signal. The main unit is configured for implementing communication functions of the wireless communication device. The conversion unit is configured for modulating a voltage from the power source according to the control signal to generate a series of electric pulses, the electric pulses ranging from 100 to 180 volts with a frequency at 0 to 20 hertz. The switch unit is configured for receiving the on signal and the electric pulses and outputting the electric pulses according to the on signal. The output unit is configured for receiving the electric pulses and applying the electric pulses to a user.

### BACKGROUND

1. Technical Field

The present disclosure relates to communication technology, and particularly, to a wireless communication device with an electric acupuncture function.

2. Description of Related Art

Wireless communication devices, such as cellular phones become popular because of their portability and necessity. Electric acupuncture equipments use electric pulses to simulate natural healing of the body. However, these electric acupuncture equipments are cumbersome and expensive.

Therefore, what is a need exists for a wireless communication device with an electric acupuncture function.

### BRIEF DESCRIPTION OF THE DRAWINGS

FIG. 1 is a schematic isometric view of a wireless communication device, according to an exemplary embodiment.

FIG. 2 is a functional block diagram of the wireless communication device of FIG. 1.

### DETAILED DESCRIPTION

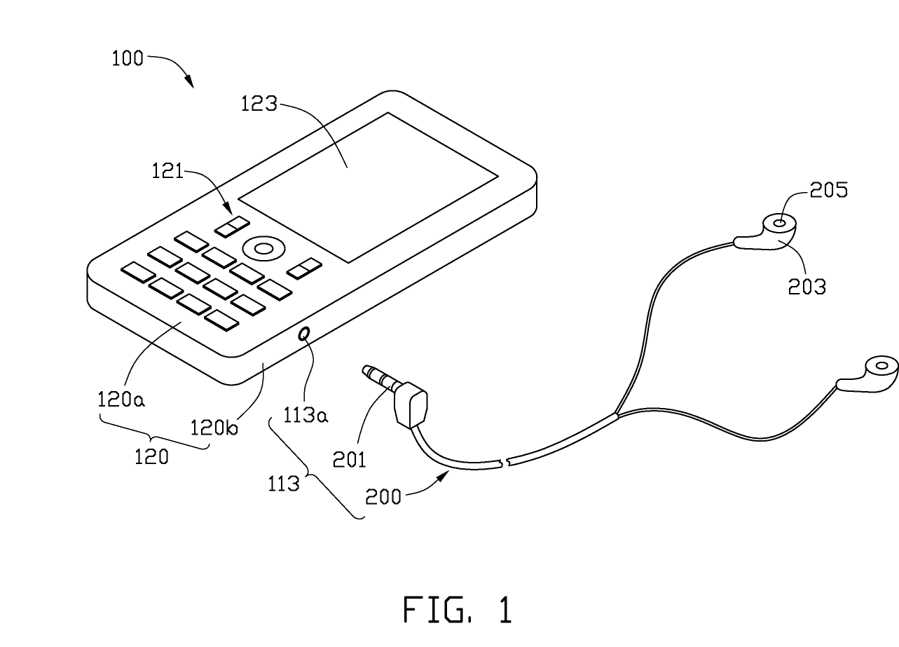

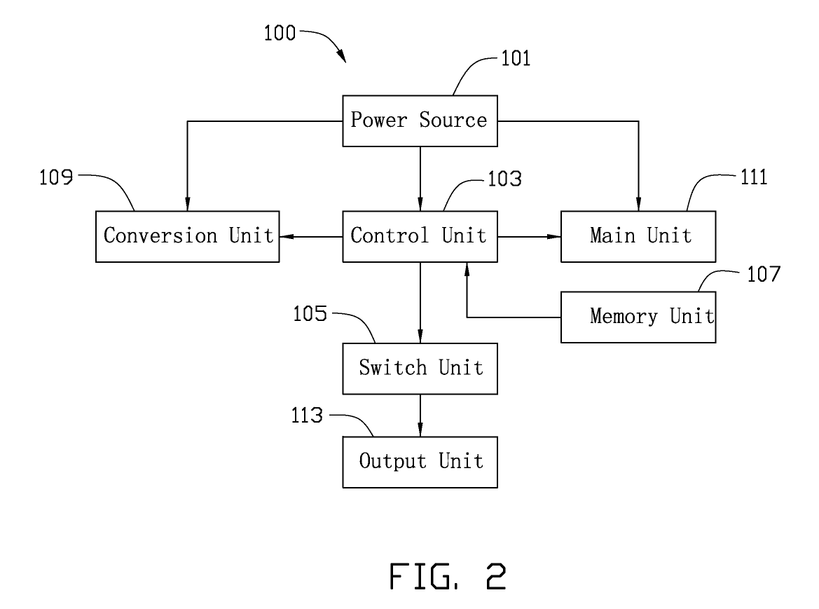

Referring to FIGS. 1 and 2, a wireless communication device 100, according to an exemplary embodiment, includes a case 120, a power source 101, a control unit 103, a switch unit 105, a memory unit 107, a conversion unit 109, a main unit 111 received in the case 120, and an output unit 113.

The case 120 includes a front sheet 120a and a side sheet 120b substantially perpendicular to the front sheet 120a. A plurality of functional keys 121 and a display 123 are separately attached to the front sheet 120a.

The power source 101 supplies power to the control unit 103, the main unit 111 and the conversion unit 109.

The memory unit 107 is configured for storing a collection of predetermined properties for the electric acupuncture. For example, the predetermined properties may include an electric pulse range, a frequency for the electric pulse, and a duration for the electric acupuncture. The memory unit 107 may be a flash memory unit.

The control unit 103 is configured for receiving the predetermined properties, generating a control signal according to the predetermined properties, and outputting an on signal to the switch unit 105. The main unit 111 is configured for implementing communication functions of the wireless communication device 100.

The conversion unit 109 is configured for modulating a voltage from the power source 101 according to the control signal to generate a series of electric pulses. In this embodiment, the electric pulses may range from 100 to 180 volts with a frequency at 0 to 20 hertz. The duration may be selected from 0 to 60 minutes.

The switch unit 105 is configured for receiving the on signal and outputting the electric pulses to the output unit 113 according to the on signal.

The output unit 113 includes a socket 113a and an attaching device 200. The socket 113a is positioned on the side sheet 120b to receive the electric pulses. The attaching device 200 is outside the case 120, and includes a plug 201 and two probes 203 electrically connected to the plug 201. The plug 201 is plugged into the socket 113a to receive the electric pulses. Each probe 203 includes an electrode 205. The two probes 203 are attached to the skin. The electric pulses are transmitted to the skin through the attaching device 200. Therefore, the wireless communication device 100 can achieve an electric acupuncture function.

It is to be understood that, in other embodiment, properties for the electric acupuncture can be inputted by a user using the functional keys 121, displayed on the display 123, and further stored in the memory unit 107. The socket 113a and the plug 201 may be omitted. The output unit 113 includes the two probes 203 directly positioned on the side sheet 120b of the case 120 with the electrodes 205 exposed from the case 120.

It is to be understood, however, that even though numerous characteristics and advantages of the present disclosure have been set forth in the foregoing description, together with details of the structure and function of the disclosure, the disclosure is illustrative only, and changes may be made in detail, especially in matters of shape, size, and arrangement of parts within the principles of the disclosure to the full extent indicated by the broad general meaning of the terms in which the appended claims are expressed.

What is claimed is:

1. A wireless communication device, comprising:
a power source;
a control unit configured for generating a control signal according to predetermined properties for an electric acupuncture and outputting an on signal;
a main unit configured for implementing communication functions of the wireless communication device;
a conversion unit configured for modulating a voltage from the power source according to the control signal to generate a series of electric pulses, the electric pulses ranging from 100 to 180 volts with a frequency at 0 to 20 hertz;
a switch unit configured for receiving the on signal and the electric pulses, and outputting the electric pulses according to the on signal; and
an output unit configured for receiving the electric pulses and applying the electric pulses to a user.

2. The wireless communication device as claimed in claim 1, wherein the predetermined properties comprise an electric pulse range, a frequency for the electric pulse, and a duration for the electric acupuncture.

3. The wireless communication device as claimed in claim 1, further comprising a memory unit configured for storing the predetermined properties.

4. The wireless communication device as claimed in claim 1, further comprising a case, the case comprising a side sheet, the output unit comprising a socket and an attaching device outside the case, the socket positioned on the side sheet, the attaching device comprising a plug and two probes electrically connected to the plug, the plug plugged into the socket to receive the electric pulses, each probe comprising an electrode.

5. The wireless communication device as claimed in claim 4, wherein the case further comprises a front sheet substantially perpendicular to the side sheet, the wireless communication device further comprising a plurality of functional keys and a display attached to the front sheet with different locations.

6. The wireless communication device as claimed in claim 1, further comprising a case, the case comprising a side sheet, the output unit comprising two probes attached to the side sheet, each probe comprising an electrode exposed from the case.

7. The wireless communication device as claimed in claim 1, wherein the wireless communication device is cellular phone.

8. The wireless communication device as claimed in claim 1, wherein the memory unit is a flash memory unit.
